(19) United States
(12) Patent Application Publication
(10) Pub. No.: US 2023/0074761 A1
(43) Pub. Date: Mar. 9, 2023

(54) INTRODUCING A CONDUCTIVE ELEMENT INTO A CATHETER

(71) Applicant: Covidien LP, Mansfield, MA (US)

(72) Inventors: Estelle J.G. Fraysse, Eindhoven (NL); Søren Aasmul, Holte (DK); Jacob D. Dove, Lafayette, CO (US)

(21) Appl. No.: 17/822,302

(22) Filed: Aug. 25, 2022

Related U.S. Application Data
(60) Provisional application No. 63/241,856, filed on Sep. 8, 2021.

(51) Int. Cl. A61M 25/06; A61M 25/00

(52) U.S. Cl. CPC A61M 25/0606; A61M 25/0097; A61M 25/06

(57) ABSTRACT

Example assemblies and techniques for introducing a conductive element into a catheter are disclosed. An example assembly includes a sensing device configured to sense a parameter of interest in a fluid, the sensing device including sensor circuitry at a distal portion, a sensing element at a proximal portion, and a conductive element communicatively coupled to the sensor circuitry and the sensing element. The assembly includes an introducer defining an introducer lumen configured to receive at least a portion of the conductive element of the sensing device, the introducer being configured to be inserted into a catheter lumen of a catheter while the at least the portion of the conductive element is in the introducer lumen. The assembly further includes a rigid member mechanically coupled to the sensing device and configured to open a longitudinal surface of the introducer as the introducer is retracted relative to the sensing device.

This application claims the benefit of U.S. Provisional Application No. 63/241,856, filed Sep. 8, 2021, the entire content of which is incorporated herein by reference.

### TECHNICAL FIELD

This disclosure relates to patient monitoring.

### BACKGROUND

Medical devices, such as catheters, may be used to assist a patient in voiding their bladder. In some instances, such catheters may be used during and/or after surgery. In the case of using a catheter to assist a patient in voiding their bladder, a Foley catheter is a type of catheter that may be used for longer time periods than a non-Foley catheter. Some Foley catheters are constructed of silicon rubber and include an anchoring member, which may be an inflatable balloon, that may be inflated in a bladder of a patient bladder to serve as an anchor so a proximal end of the catheter does not slip out of the bladder of the patient.

### SUMMARY

In general, the disclosure describes devices and assemblies configured to facilitate the introduction of a conductive element, such as an electrically conductive wire or an optical fiber, into a lumen of a catheter, and techniques for using the devices and assemblies. The conductive element can be, for example, part of a sensing device configured to sense a parameter of interest in a fluid (e.g., urine) during patient monitoring, such as renal monitoring. In the case of renal monitoring (also referred to herein as kidney function monitoring), the conductive element can be configured to be introduced into a lumen of a Foley catheter, e.g., to position at least part of the sensing device in the bladder of a patient, in a fluid pathway from the bladder to a location external to the patient, or the like.

In one example, this disclosure describes an assembly including a sensing device configured to sense a parameter of interest in a fluid, the sensing device comprising sensor circuitry at a distal portion, a sensing element at a proximal portion, and a conductive element communicatively coupling the sensor circuitry and the sensing element; an introducer defining an introducer lumen configured to receive at least a portion of the conductive element of the sensing device, the introducer being configured to be at least partially inserted into a catheter lumen of a catheter while the at least the portion of the conductive element is in the introducer lumen; and a rigid member mechanically coupled to the sensing device and configured to open a longitudinal surface of the introducer as the introducer is retracted relative to the sensing device.

In one example, this disclosure describes a method including retracting an introducer from a catheter lumen of a catheter, the introducer defining an introducer lumen configured to receive at least a portion of a conductive element of a sensing device, the introducer being configured to be at least partially inserted into the catheter lumen while the at least the portion of the conductive element is in the introducer lumen, wherein a rigid member is mechanically coupled to the sensing device and is configured to open a longitudinal surface of the introducer as the introducer is retracted relative to the sensing device; and removing the introducer from the catheter lumen.

In another example, this disclosure describes an assembly including a catheter defining a catheter lumen; a sensing device configured to sense oxygen content in a fluid, the sensing device comprising sensor circuitry at a distal portion, a sensing element at a proximal portion, and an optical fiber communicatively coupled to the sensor circuitry and the sensing element; an introducer defining an introducer lumen configured to receive at least a portion of the optical fiber, the introducer being at least partially disposed within the catheter lumen and being configured to be retracted from the catheter lumen while the at least the portion of the optical fiber is in the introducer lumen; and a rigid member mechanically coupled to the sensing device and configured to open a longitudinal surface of the introducer as the introducer is retracted relative to the sensing device.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is a conceptual side elevation view of an example catheter.

FIG. 2 is a diagram illustrating an example cross-section of an elongated body of a catheter, where the cross-section is taken along line 2-2 in FIG. 1.

FIG. 3 is a block diagram of an example external device that may be used with a medical device.

FIG. 4 is a graph illustrating pO₂ measurements of water that was initially set to ˜43 mmHg and allowed to flow through a silicone Foley catheter at different flow rates.

FIG. 5 is a conceptual diagram illustrating a perspective view of an example assembly inserted into a lumen of a catheter.

FIG. 6 is a conceptual cross-sectional view of an example distal portion of a catheter having the assembly of FIG. 5 inserted within a catheter lumen, the cross-section being taken along a longitudinal axis of the catheter.

FIG. 7 is a conceptual exploded view of a distal portion of the assembly of FIG. 5.

FIGS. 8A and 8B are alternative planar views of an example introducer.

FIG. 9 is a conceptual cross-sectional diagram of a distal portion of an example assembly inserted within a catheter lumen of a catheter, the cross-section being taken along a longitudinal axis of the catheter.

FIG. 10 is a conceptual diagram of a proximal portion of an example assembly within a catheter lumen of a catheter.

FIG. 11 is a conceptual diagram of an example conductive element and sensing element within a catheter lumen after an introducer catheter is retracted from the catheter lumen.

FIG. 12 is a conceptual diagram illustrating an example ring configured to help prevent a sensing element from contacting a surface defining a catheter lumen.

FIG. 13 is a flowchart illustrating example assembly usage techniques of this disclosure.

### DETAILED DESCRIPTION

Acute kidney injury (AKI) is a complication that may occur after some medical procedures, such as some cardiac surgeries, e.g., coronary artery bypass grafting (CABG). AKI may also occur after other surgeries that are lengthy and involve significant blood loss or fluid shifts. For example, a body of a surgery patient may alter where their blood is directed which may lead to hypoxia of a kidney. A cause of surgery-associated AKI is hypoxia of the kidneys, which may cause an ischemia reperfusion injury in a kidney of the patient. This ischemia reperfusion injury may cause degradation of renal function of the patient. The degradation of renal function may cause an accumulation of waste products in the bloodstream, which may delay the recovery of the patient from the surgery and lead to more extended hospital stays and may even lead to further complications.

The present disclosure describes example devices that are configured to facilitate the monitoring of kidney function of patients, such as patients who are undergoing or who have undergone such surgeries, based on a parameter of interest (e.g., oxygen content) of a fluid (e.g., urine) in or removed from the patient. The monitoring of kidney function may help reduce occurrences of AKI by providing clinicians with an assessment of the risk that a specific patient may develop AKI. This may facilitate a clinician intervening prior to the patient developing AKI. For example, a clinician may initiate or make changes to hemodynamic management (e.g., blood pressure management, fluid management, blood transfusions, and the like), make changes to cardiopulmonary bypass machine settings, or avoid providing nephrotoxic drugs. Post operatively, a clinician may intervene with a Kidney Disease: Improving Global Outcomes (KDIGO) bundle or an AKI care bundle. While urine, bladders, and AKI are primarily referred to herein to describe the example devices, in other examples, the devices may be used with other target locations in a patient, such as intravascular locations, and to monitor fluids of interest other than urine and/or other patient conditions other than kidney function.

While systemic vital signs like cardiac output, blood pressure, and hematocrit may be useful for monitoring the kidney function of a patient (also referred to herein as renal monitoring), it may also be useful to monitor the oxygenation status of the kidneys in order to limit, reduce the severity of, or even prevent the risk of AKI. Accurate monitoring of the oxygenation status of the kidneys can be challenging due to the inaccessibility of the kidneys. Near-Infrared spectroscopy (NIRS) measures regional oximetry, and has some utility in babies and relatively slender adults in measuring oxygenation of the kidneys, but may not have the depth of penetration and specificity required for some patients.

In some examples, it may be desirable to sense a parameter of interest in a fluid close to the kidneys as the parameter may change the further removed the fluid (e.g., both time and distance) is from the kidneys. It may not be feasible to include an entire sensor on or in a proximal portion of a catheter as that may cause the catheter to become stiff or be too large to be inserted into the urethra of a patient. Therefore, it may be desirable to use a sensing device having a sensing element at a proximal end, sensor circuitry at a distal end (e.g., external to the patient), and a conductive element (such as an optical fiber or an electrical wire) communicatively coupling the sensing element and the sensor circuitry. Introducing a relatively thin conductive element into a catheter lumen may be challenging due the flexibility of the conductive element, which may have a relatively low column strength and flexural rigidity, and therefore may exhibit relatively low pushability (e.g., through the catheter lumen). In addition, the catheter may be formed from a material that is slightly sticky (e.g., silicone, latex or polyvinyl chloride (PVC)), such that there is some friction between the conductive element and the surface of the catheter defining the catheter lumen. As a result, the conductive element may buckle during insertion of the conductive element directly into the catheter lumen, hindering the insertion of the sensing device into the catheter lumen.

The present disclosure describes example assemblies configured to facilitate insertion of a sensing device into a catheter lumen of a catheter. In some examples, the assemblies include a sensing device, an introducer, and a rigid member. In addition, in some examples, the assemblies may include the catheter. The introducer is configured to facilitate the introduction of the conductive element, such as an electrically conductive wire or an optical fiber, into a lumen of a catheter. For example, the conductive element may be relatively flexible and, therefore, difficult to push directly into the catheter lumen, and the introducer may be more rigid than the conductive element and positioned around the conductive element to help facilitate introduction of the conductive element into the catheter lumen. The conductive element can be, for example, part of a sensing device configured to sense a parameter of interest in a fluid (e.g., urine) during patient monitoring, such as renal monitoring. In the case of renal monitoring, the conductive element can be configured to be introduced into a lumen of a Foley catheter, e.g., to position at least part of the sensing device in the bladder of a patient, in a fluid pathway from the bladder to a location external to the patient, or the like.

The rigid member is configured open a longitudinal surface of the introducer as the introducer is retracted relative to the sensing device. This facilitates removal of the introducer from around the conductive element without, for example, causing a sensing element of the sensing device to move or without causing the sensing element to move from a desired target position in the patient.

The sensing element can be configured to sense any suitable parameter of interest in a fluid. In some examples, sensing device includes a sensing element configured to sense an amount of dissolved oxygen, such as the oxygen partial pressure, in a fluid, such as urine in the case of kidney function monitoring. The sensing element may also be configured to provide a signal indicative of the amount of dissolved oxygen in the fluid to sensor circuitry via a conductive element of the sensing device. The sensing circuitry may include electrical, opto-electrical, and/or optical components.

While urine, bladders, and AKI are primarily referred to herein to describe the example assemblies, in other examples, the assemblies may be used with other target locations in a patient, such as intravascular locations, and to monitor fluids of interest other than urine and/or other patient conditions other than kidney function. As discussed in further detail below, in some examples, the sensing element may include a dissolved oxygen sensor configured to sense an amount of oxygen dissolved in the urine (e.g., oxygen partial pressure pO₂) in the bladder or in the catheter, from which a clinician or a device may be able to determine an oxygenation status of the one or both kidneys of the patient.

While the sensing device of this disclosure is primarily described as sensing an amount of dissolved oxygen in a fluid, other parameters of interest may be sensed by the sensing device. These other parameters of interest may include, but are not limited to, any one or more of temperature, pressure, urine flow rate, urine concentration, urine electrical conductivity, urine specific gravity, urine biomarkers, amount of dissolved carbon dioxide in the urine, urine pH, bladder or abdominal pressure, urine color, urine turbidity, urine creatinine, urine electrical conductivity, urine sodium, or motion. In some cases, it may be desirable to sense one or more of these parameters relatively close to the kidneys as possible because when sensors are positioned further away from the kidneys, the risk of introducing noise or losing signal strength increases and/or the risk of the concentration or integrity of a substance of interest in the fluid of interest (e.g., urine) changing prior to being sensed by the sensing device may increase.

In the case of a Foley catheter, it may be desirable to sense the amount of dissolved oxygen in the fluid and/or one or more of the other parameters listed above at a proximal portion of the Foley catheter (e.g., in the bladder of the patient). However, placing these sensors at the proximal portion of the catheter may increase the size and stiffness of the catheter and, as a result, may undermine the patient comfort or deliverability of the catheter. By design, a Foley catheter is configured to be relatively small and flexible, such that it can be inserted through the urethra and into the bladder of a patient. If a Foley catheter were stiffer or include sensors disposed on an outer surface of the catheter, then it may be more difficult to comfortably insert the catheter into the bladder of the patient.

As used herein, “sense” may include detect and/or measure.” As used herein, “proximal” is used as defined in Section 3.1.4 of ASTM F623-19, Standard Performance Specification for Foley Catheter. That is, a proximal end of a catheter is the end closest to the patient when the catheter is being used by the patient. The distal end is therefore the end furthest from the patient. In some examples, “prevent” may mean completely prevent or partially prevent (e.g., effectively prevent), such as by blocking, restricting, inhibiting, impeding, or hindering.

The amount of dissolved oxygen in a urine of a patient may be indicative of kidney function or kidney health. For example, dissolved oxygen in urine of a patient in the bladder may correlate to perfusion and/or oxygenation of the kidneys, which is indicative of kidney performance. However, dissolved oxygen can be relatively difficult to measure. One way to measure dissolved oxygen is by fluorescence or luminescence lifetime sensor(s). For example, a sensing element may be a portion of or all of a fluorescence or luminescence lifetime sensor. The sensing element may utilize a light which may originate from sensor circuitry and sense the decay of glow from the light in a fluid, which may be indicative of the level of oxygen in the fluid. To more accurately measure the level of oxygen in urine of a patient, it may be desirable to take the measurement prior to any significant modification in the oxygen content in the urine, e.g., as close to the kidneys as possible. However, it may not be feasible to place all of a sensing device at the proximal end of the catheter as doing so may increase cost, size, and decrease flexibility of the catheter.

Some Foley catheters include an elongated body made from a silicone rubber that is relatively permeable to oxygen. Thus, as a fluid flows through a drainage lumen of the Foley catheter from a proximal fluid opening through the drainage lumen to a distal fluid opening to the drainage lumen, some oxygen may permeate from the surrounding environment through the walls of the elongated body into urine in the drainage lumen or dissipate through the walls of the elongated body and into a surrounding environment. For example, urine oxygenation for some patients may be 10 millimeters of mercury (mmHg) to 50 mmHg, which is substantially lower than the atmospheric level of about 150 mmHg, creating a gradient that can drive atmospheric oxygen into the catheter.

In accordance with examples of this disclosure, an assembly includes a sensing device configured to sense a parameter of interest in a fluid, the sensing device comprising sensor circuitry at a distal portion, a sensing element at a proximal portion, and a conductive element communicatively coupling the sensor circuitry and the sensing element. The conductive element is configured to extend through a catheter lumen and enables the sensing element to be position in or relatively close to the bladder (or other fluid source) and the sensor circuitry to be positioned external to the patient. In the case of monitoring the oxygen content of urine, the sensing element may sense the dissolved oxygen in urine relatively close to a bladder of a patient or in the bladder of the patient. Enabling the sensing circuitry to remain outside the patient can enable the Foley catheter to retain a relatively low profile for patient comfort.

The assembly further includes an introducer defining an introducer lumen configured to receive at least a portion of the conductive element of the sensing device, the introducer being configured to be inserted into a catheter lumen of a catheter while the at least the portion of the conductive element is in the introducer lumen. The assembly also includes a rigid member, such as a hollow needle, mechanically coupled to the sensing device and configured to open a longitudinal surface of the introducer as the introducer is retracted relative to the sensing device.

The assembly may enable a clinician to insert the sensing element and conductive element into the catheter lumen even though the conductive element may be relatively flexible and have low column strength. In some examples, the clinician may insert a proximal portion of the assembly into the catheter lumen after the catheter is inserted into the urethra of a patient. This may enable the catheter to remain relatively flexible as it is introduced into the patient, thereby reducing patient discomfort which may otherwise be caused by inserting a catheter having an oxygen sensor already contained within the catheter lumen.

In some examples, the sensing element is separate from and configured to be introduced into the catheter lumen of the catheter, which may enable the catheter to remain relatively flexible, e.g., compared to examples in which the oxygen sensing element and associated wires or fiber optic elements is integrated into the catheter. The flexibility may help maintain the deliverability of the Foley catheter proximal end to the bladder.

The sensing element may be configured to sense an amount of dissolved oxygen in a fluid (e.g., urine) and provide a signal indicative of the amount of dissolved oxygen in the fluid to the sensor circuitry. The sensing element may be located in a proximal portion of a catheter lumen of a catheter. The sensor circuitry may be located at a distal portion of the catheter lumen or distal to a distal end of the catheter lumen.

In some examples, the catheter is a Foley catheter, which defines a drainage lumen. The drainage lumen is configured to facilitate the flow of the fluid from a first fluid opening at a proximal end of the catheter to a second fluid opening at a distal end of the opening, e.g., from a bladder to a collection container outside of a patient. In some examples, the assembly is configured to be inserted into the drainage lumen. In other examples, the assembly is configured to be inserted into a lumen other than a drainage lumen of the Foley catheter.

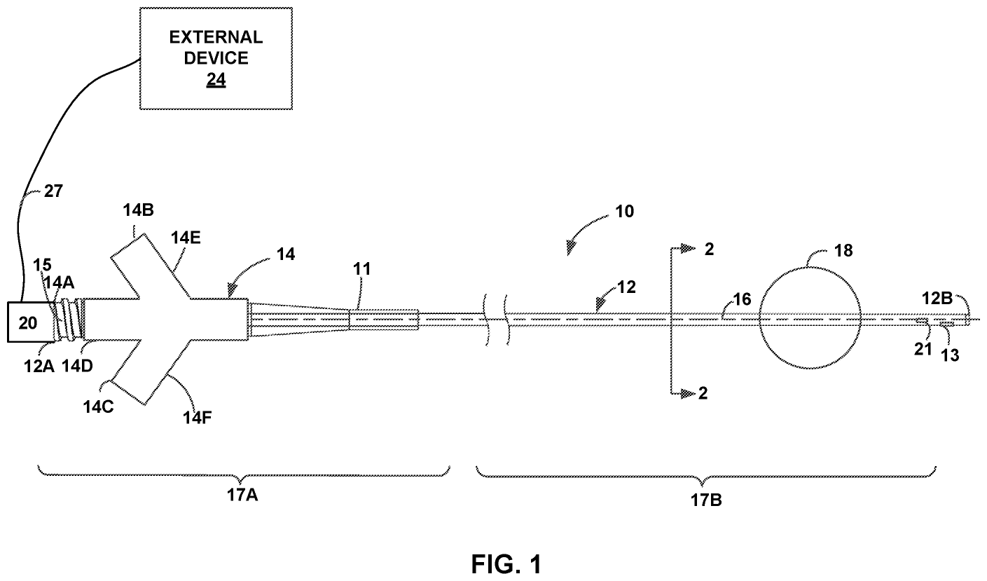

FIG. 1 is a conceptual side elevation view of an example catheter. Catheter 10 which includes elongated body 12, hub 14, and anchoring member 18. In some examples, catheter 10 is a Foley catheter. While a Foley catheter and its intended use are primarily referred to herein to describe catheter 10, in other examples, catheter 10 can be used for other purposes, such as to drain wounds or for intravascular monitoring or medical procedures.

Catheter 10 includes a distal portion 17A and a proximal portion 17B. Distal portion 17A includes a distal end 12A of elongated body 12 and is intended to be external to a body of a patient when in use, while proximal portion 17B includes a proximal end 12B of elongated body 12 and is intended to be internal to a body of a patient when in use. For example, when proximal portion 17B is positioned within a patient, e.g., such that proximal end 12B of elongated body 12 is within the bladder of a patient, distal portion 17A may remain outside of the body of the patient.

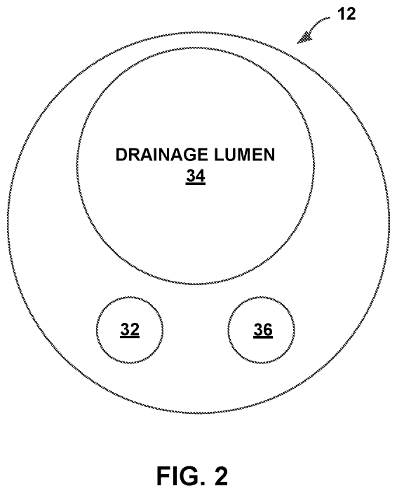

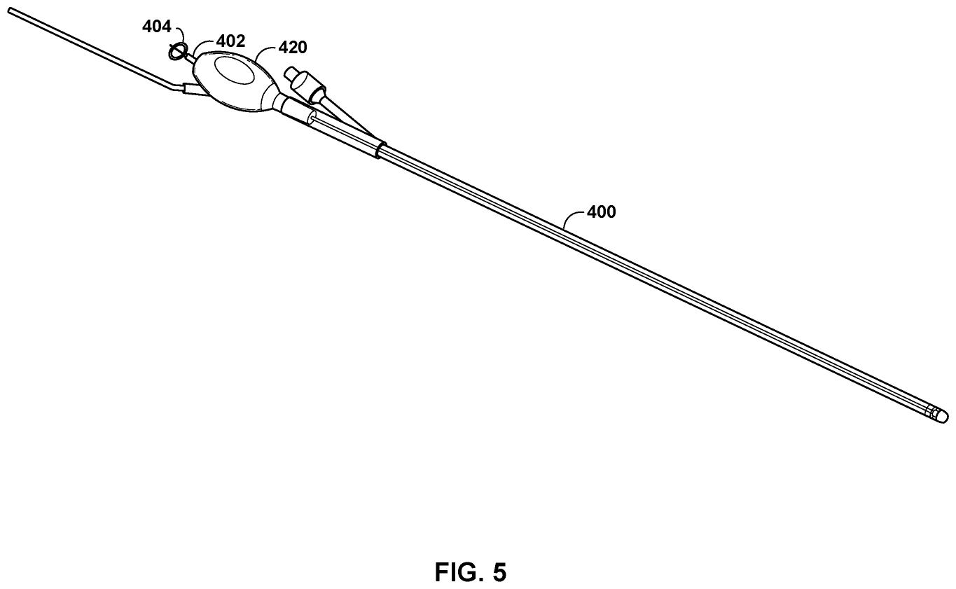

Elongated body 12 is a structure (e.g., a tubular structure) that extends from distal end 12A to proximal end 12B and defines one or more inner lumens. In the example shown in FIGS. 1-2, elongated body 12 defines lumen 32, drainage lumen 34, and anchoring lumen 36 (shown in FIG. 2). In some examples, drainage lumen 34 is configured to drain a fluid from a target site, such as a bladder. In other examples, drainage lumen 34 may be used for any other suitable purpose, such as to deliver a substance or another medical device to a target site within a patient. Drainage lumen 34 may extend from proximal fluid opening 13 to distal fluid opening 14A. Both proximal fluid opening 13 and distal fluid opening 14A may be fluidically coupled to drainage lumen 34, such that a fluid may flow from one of fluid opening 13 or fluid opening 14A to the other of fluid opening 13 or fluid opening 14A through drainage lumen 34. Fluid opening 13 and fluid opening 14A may also be referred to as drainage openings. In some examples, drainage lumen 34 may be configured to receive or house at least a portion of an assembly including a sensing device, an introducer, and a rigid member. The assembly is discussed in more detail later in this disclosure with respect to FIGS. 5-11. In some examples, lumen 32 (shown in FIG. 2) may be configured to receive or house at least a portion of the assembly. In this manner, lumen 32 may be referred to as a sensor lumen.

Sensing element 21 and sensor circuitry 20 are depicted. Sensing element 21 and sensor circuitry 20 may be part of a sensing device which may be part of the assembly and may be communicatively coupled together via a conductive element (not shown in FIG. 1). In some examples, the conductive element is an electrical wire configured to conduct an electrical signal and/or an optical fiber configured to conduct an optical signal.

In some examples, sensing element 21 includes an oxygen sensing element. For example, sensing element 21 may include an oxygen sensitive luminophore configured to be excited optically and emit luminescent light with an intensity and decay time inversely proportional to the oxygen concentration in a fluid in a lumen of catheter 10 or external to the lumen. The excitation and emission light may be transmitted via a conductive element (e.g., an optical fiber) between the oxygen sensitive indicator and an interrogating optical system (e.g., sensor circuitry 20). The oxygen sensitive indicator may be located at or near the tip of the conductive element which may be placed at or near the proximal end of catheter 10 such as to be in the bladder of the patient and in direct contact with the urine within the lumen whereas sensor circuitry 20 is at distal end 12A or distal to the distal end of catheter 10.

In some examples, lumen 32 extends from fluid opening 14C to a location proximate to anchoring member 18, such as to proximal end 12B. In some examples, there may be a fluid opening at proximal end 12B fluidically coupled to lumen 32 and configured to enable the flow of a fluid from, for example, a bladder of a patient into lumen 32.

For example, sensor circuitry 20 may be at a distal portion of the sensing device and sensing element 21 may be at a proximal portion of the sensing device. The sensing device may be part of an assembly that may be configured to be introduced into lumen 32 via fluid opening 14C or drainage lumen 34 via fluid opening 14A. Sensor circuitry 20 may include optical, optoelectrical, and/or electrical components and may be configured to determine an amount of dissolved oxygen in a fluid based on a signal received from sensing element 21 via the conductive element. For example, the sensing device may include a fluorescence or luminescence lifetime sensor(s) and sensing element 21 may receive light from sensor circuitry 20 via the conductive element, may focus that light towards a fluid and sense the decay of the glow caused by the light. Sensing element 21 may communicate a signal indicative of the amount of dissolved oxygen in the fluid to sensor circuitry 20 via the conductive element.

In some examples, sensor circuitry 20 is communicatively coupled to external device 24 and be configured to provide a signal indicative of the amount of dissolved oxygen in the fluid to processing circuitry of external device 24 via connection 27. External device 24 may be a computing device, such as a workstation, a desktop computer, a laptop computer, a smart phone, a tablet, a server or any other type of computing device that may be configured to receive, process and/or display sensor data. Connection 27 may be an electrical, optical, wireless or other connection.

Proximal portion 17B of catheter 10 comprises anchoring member 18, fluid opening 13, and sensing element 21. In some examples, sensing element 21 is received or housed within lumen 32. In other examples, sensing element 21 may be configured to extend external to elongated body 12, e.g., extend proximally from proximal end 12B of elongated body 12. Fluid opening 13 may be positioned on the surface of elongated body 12 between anchoring member 18 and the proximal end 12B (as shown) or may be positioned at the proximal end 12B.

Anchoring member 18 may include any suitable structure configured to expand from a relatively low profile state to an expanded state in which anchoring member 18 may engage with tissue of a patient (e.g., inside a bladder) to help secure and prevent movement of proximal portion 17B out of the body of the patient. For example, anchoring member 18 can include an anchor balloon or other expandable structure. When inflated or deployed, anchoring member 18 may function to anchor catheter 10 to the patient, for example, within the bladder of the patient. In this manner, the portion of catheter 10 on the proximal side of anchoring member 18 may not slip out of the bladder of the patient.

Anchoring lumen 36 (shown in FIG. 2) may be configured to transport a fluid, such as sterile water or saline, or a gas, such as air, from distal opening 14B to anchoring member 18. For example, an inflation device (not shown) may pump fluid or gas into anchoring lumen 36 through distal opening 14B into anchoring member 18 such that anchoring member 18 is inflated to a size suitable to anchor catheter 10 within the bladder of the patient. In examples in which anchoring member 18 does not include an expandable balloon, anchoring lumen 36 may be configured to receive a deployment mechanism (e.g., a pull wire or a push wire) for deploying an expandable structure anchoring member 18 and hub 14 may comprise distal fluid opening 14A, fluid opening 14C and a distal opening 14B via which a clinician may access the deployment mechanism.

In some examples, such as examples in which catheter 10 is a Foley catheter, elongated body 12 has a suitable length for accessing the bladder of a patient through the urethra. The length may be measured along central longitudinal axis 16 of elongated body 12. In some examples, elongated body 12 may have an outer diameter of about 12 French to about 14 French, but other dimensions may be used in other examples. Distal portion 17A and proximal portion 17B of elongated body 12 may each have any suitable length.

In the example shown in FIG. 1, distal end 12A of elongated body 12 is received within hub 14 and is mechanically connected to hub 14 via an adhesive, welding, or another suitable technique or combination of techniques. Hub 14 is positioned at a distal end of elongated body 12 and defines an opening through which the one or more inner lumens (e.g., lumen 32, drainage lumen 34 and anchoring lumen 36, shown in FIG. 2) of elongated body 12 may be accessed and, in some examples, closed. While hub 14 is shown in FIG. 1 as having three arms, 14D, 14E and 14F, hub 14 may have any suitable number of arms, which may, in some examples, depend on the number of inner lumens defined by elongated body 12. For example, each arm may be fluidically coupled to a respective inner lumen of elongated body 12. In the example of FIG. 1, hub 14 comprises a distal fluid opening 14A, which is fluidically coupled to drainage lumen 34, a distal opening 14B, which is fluidically coupled to anchoring lumen 36, and fluid opening 14C which is fluidically coupled to lumen 32 (shown in FIG. 2) of elongated body 12. In examples in which anchoring member 18 does not include an expandable balloon, anchoring lumen 36 may be configured to receive a deployment mechanism (e.g., a pull wire or a push wire) for deploying an expandable structure anchoring member 18.

In examples in which catheter 10 is a Foley catheter, a fluid collection container (e.g., a urine bag) may be attached to distal fluid opening 14A for collecting urine draining from the bladder of the patient. Distal opening 14B may be configured to connect to an inflation device to inflate anchoring member 18 positioned on proximal portion 17B of catheter 10. Anchoring member 18 may be uninflated or undeployed when not in use. Hub 14 may include connectors, such as connector 15, for connecting to other devices, such as the fluid collection container and the inflation source. Fluid opening 14C may be operable to receive or house an assembly including a sensing device that may include sensing element 21, which may be configured to sense a parameter of interest in a fluid, such as oxygen, an introducer and a rigid member. In some examples, catheter 10 includes strain relief member 11, which may be a part of hub 14 or may be separate from hub 14.

In some examples, sensor circuitry 20 is positioned distal to distal end 12A, such as attached to connector 15, on additional tubing, or attached to another structure connected to hub 14.

Although sensor circuitry 20 and sensing element 21 of a sensing device are shown in FIG. 1, in some examples, catheter 10 can include any suitable number of sensors on proximal portion 17B and/or any suitable number of sensors on distal portion 17A, where the sensors on proximal portion 17B sense the same or different parameters and the sensors on distal portion 17A sense the same or different parameters. In addition, some or all of the sensors on proximal portion 17B may sense the same or different parameters as the sensors on distal portion 17A. For example, in the case where sensors on the distal portion may be temperature dependent, it may be desirable to sense temperature both on the proximal portion 17B and the distal portion 17A.

Elongated body 12 may be structurally configured to be relatively flexible, pushable, and relatively kink- and buckle- resistant, so that it may resist buckling when a pushing force is applied to a relatively distal portion of the medical device to advance the elongated body proximally through the urethra and into the bladder. Kinking and/or buckling of elongated body 12 may hinder a clinician's efforts to push the elongated body proximally.

In some examples, at least a portion of an outer surface of elongated body 12 includes one or more coatings, such as an anti-microbial coating, and/or a lubricating coating. The lubricating coating may be configured to reduce static friction and/ kinetic friction between elongated body 12 and tissue of the patient as elongated body 12 is advanced through the urethra.

FIG. 2 is a diagram illustrating an example cross-section of an elongated body of a catheter, where the cross-section is taken along line 2-2 in FIG. 1 in a direction orthogonal to central longitudinal axis 16. FIG. 2 depicts a cross section of elongated body 12, which defines lumen 32, drainage lumen 34, and anchoring lumen 36. While lumen 32, drainage lumen 34, and anchoring lumen 36 are shown as circular in cross-section, they may have any suitable cross-sectional shape in other examples.

Elongated body 12 may define any suitable number of lumens. For example, although one anchoring lumen 36 is shown in FIG. 2, in other examples, elongated body 12 can define a plurality of anchoring lumens 36, e.g., that are distributed around lumen 32 or drainage lumen 34. As another example, anchoring member 18 may be an expandable structure that is not an inflatable balloon. In such examples, anchoring lumen 36 may be replaced by or house a deployment mechanism which may permit a clinician to expand the expandable structure. For example, anchoring lumen 36 may be replaced by or house a mechanical device that may be pushed and pulled separately from the catheter 10 by a clinician to expand or retract the expandable structure.

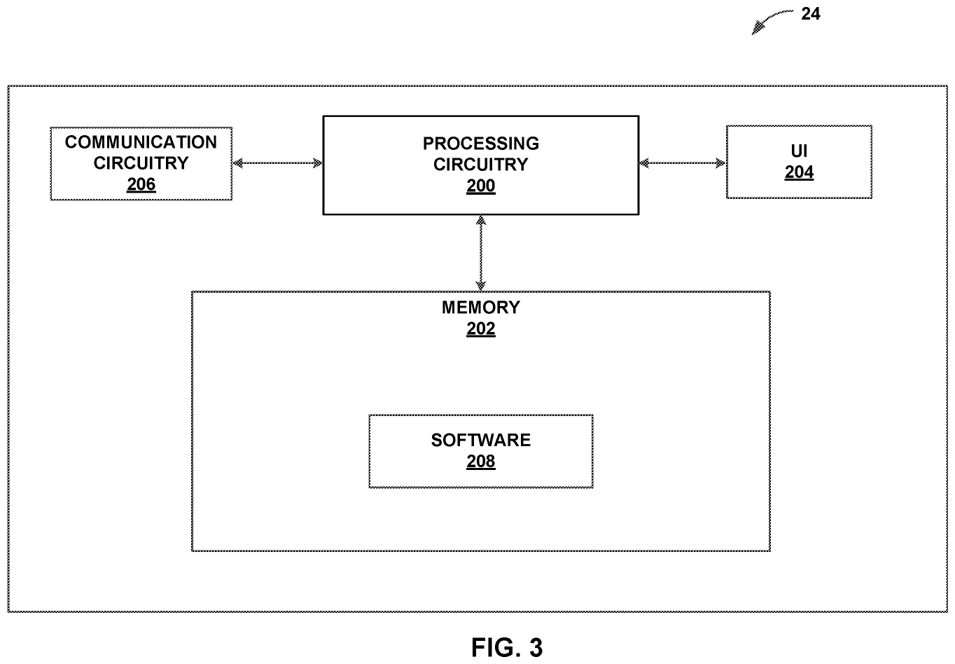

FIG. 3 is a functional block diagram illustrating an example of an external device 24 that may be used with a medical device. In the example of FIG. 3, external device 24 includes processing circuitry 200, memory 202, user interface (UI) 204, and communication circuitry 206. External device 24 may be a dedicated hardware device with dedicated software for the reading sensor data. Alternatively, external device 24 may be an off-the-shelf computing device, e.g., a desktop computer, a laptop computer, a tablet, or a smartphone running a mobile application that enables external device 24 to read sensor data from sensor circuitry 20.

In some examples, a user of external device 24 may be clinician. In some examples, a user uses external device 24 to monitor kidney function of a patient. In some examples, the user may interact with external device 24 via UI 204, which may include a display to present a graphical user interface to the user and/or sound generating circuitry configured to generate audio output, and a keypad or another mechanism (such as a touch sensitive screen) configured to receive input from the user. External device 24 may communicate with sensor circuitry 20 using wired, wireless or optical methods through communication circuitry 206. For example, processing circuitry 200 of external device 24 may process sensor data from sensor circuitry 20.

Processing circuitry 200 may include any combination of integrated circuitry, discrete logic circuity, analog circuitry, such as one or more microprocessors, digital signal processors (DSPs), application specific integrated circuits (ASICs), or field-programmable gate arrays (FPGAs). In some examples, processing circuitry 200 may include multiple components, such as any combination of one or more microprocessors, one or more DSPs, one or more ASICs, or one or more FPGAs, as well as other discrete or integrated logic circuitry, and/or analog circuitry.

Memory 202 may store program instructions, such as software 208, which may include one or more program modules, which are executable by processing circuitry 200. When executed by processing circuitry 200, such program instructions may cause processing circuitry 200, and external device 24 to provide the functionality ascribed to them herein. The program instructions may be embodied in software and/or firmware. Memory 202 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media.

This disclosure describes techniques and devices configured to aid in the monitoring of the one or both kidneys of a patient. In some examples, processing circuitry 200 of external device 24 monitors the amount of oxygen dissolved in the urine (pO₂) in the bladder as it has been shown that this measurement reflects the oxygenation of the kidneys. To do this the amount of oxygen dissolved in the urine may be sensed by sensing element 21.

Patients can be catheterized during and after major surgery using an indwelling urinary (e.g., Foley) catheter (e.g., catheter 10) inserted into the bladder via the urethra. Oxygen may be sensed at the distal portion 17A of catheter 10 or distal to distal end 12A using a sensing device. However, as mentioned above, commercially available Foley catheters are oxygen permeable in varying degrees depending on the material from which the catheter was constructed. This results in diffusion of oxygen between the urine in catheter 10 and the ambient air as well as the urethra over the catheter wall. Furthermore, the catheter wall constitutes an oxygen buffer which takes up/releases oxygen from/to the urine. These mechanisms result in an alteration of the pO₂ from the true value in the bladder over the length of the catheter to the sample point at or near distal end 12A of catheter 10 where sensing element 20 senses the pO₂.

Due to the relatively high oxygen permeability of Foley catheters, it may be desirable for a measurement of urine oxygenation to be taken as close to the kidneys as possible to obtain the best signal, e.g., more accurate reading indicative of the oxygenation status of the kidneys. However, it may not be easy to place a sensor through the ureter, so an alternative location to use is in the bladder. The measurements may also be taken outside of the body, but the urine transit through a Foley catheter has the possibility of changing the measurement. For example, the Foley catheter may be formed from silicone, which has a relatively high permeability to oxygen. In some cases, the urine oxygenation is in the range of 10 to 50 mmHg, which is substantially lower than the atmospheric level of about 159 mmHg at sea level, creating a gradient that may drive atmospheric oxygen into the lumens of the Foley catheter. To make a more accurate measurement of the oxygen content of urine, it may be preferable to take the measurement in the bladder or relatively close to the bladder.

Example assemblies described herein are configured to facilitate introduction of a conductive element of the oxygen sensor or other sensor into a catheter lumen (e.g., lumen 32 or drainage lumen 34). In some examples, a user (e.g., a clinician) introduces the conductive element of the sensing device into the catheter lumen. In other examples, the conductive element is prepositioned, e.g., by a manufacturer, in the catheter lumen before the system is used to monitor a patient.

In some examples, the assembly includes a sensing device, an introducer, and a rigid member. The introducer defines an introducer lumen configured to receive at least a portion of a conductive element of the sensing device and is configured to be at least partially inserted into the catheter lumen of catheter 10 while the at least the portion of the conductive element is in the introducer lumen. In some examples, the rigid member is mechanically coupled to the sensing device and configured to open a longitudinal surface of the introducer as the introducer is retracted from the catheter lumen relative to the sensing device to facilitate separation of the introducer from the sensing element while leaving the conductive element in place within the catheter lumen.

In this manner, a clinician may select a commercially available Foley catheter of their choice and use an assembly including the sensing device, the introducer and the rigid member to introduce the proximal portion of the sensing device into the catheter lumen. In some examples, the assembly may include the catheter and the clinician may introduce the proximal portion of the catheter into the urethra of the patient before removing the inserter from the catheter lumen.

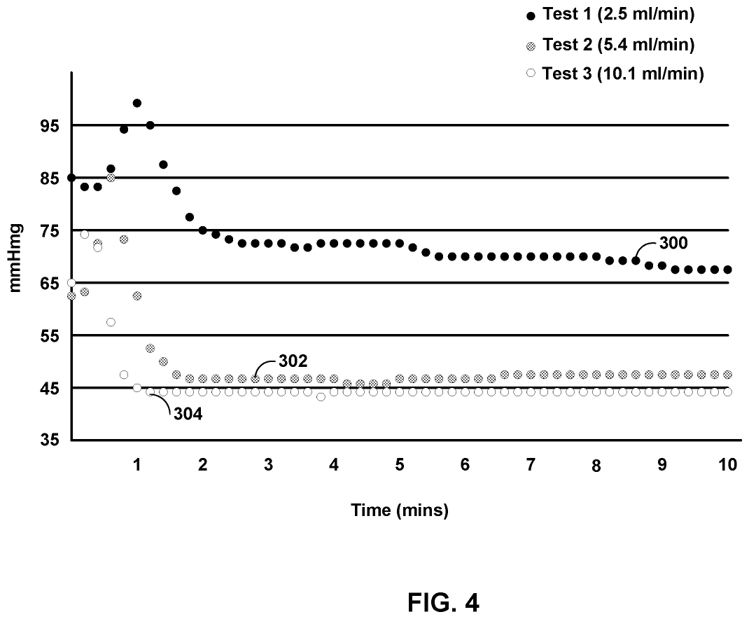

FIG. 4 is a graph illustrating pO₂ measurements of water that was initially set to ˜43 mmHg and allowed to flow through a silicone Foley catheter at different flow rates. As shown in FIG. 4, the O₂ pick up from water with an initial pO₂ of ˜43 mmHg flowing through a silicone Foley Catheter at a flow rate of 2.5 ml/min (Test 1 whose measurements shown as black filled circles 300) measured a pO₂ uptake of 26.3 mmHg. The O₂pick up from water with an initial pO₂ of ˜43 mmHg flowing through a silicone Foley Catheter at a flow rate of 5.4 ml/min (Test 2 whose measurements shown as grey filled circles 302) measured a pO₂ uptake of 6.4 mmHg. The O₂ pick up from water with an initial pO₂ of ˜43 mmHg flowing through a silicone Foley Catheter at a flow rate of 10.1 ml/min (Test 3 whose measurements shown as white filled circles line 304) measured a pO₂ pick up of 3 mmHg. Some urine flow rates range from 0-5 ml/min for catheterized patients. Hence, the pO₂ pick up could be significant for silicone catheters.

FIG. 5 is a conceptual diagram illustrating a perspective view of an example assembly inserted in a lumen of a catheter. Introducer 402 is shown inserted through a proximal portion of a sensing device including sensor circuitry 420, where the sensing device is introduced in a catheter lumen (not shown in FIG. 5) of catheter 400. Catheter 400 may be an example of catheter 10 of FIG. 1 and the catheter lumen can be lumen 32 or drainage lumen 34 of catheter 10 in various examples. In this example, catheter 400 is depicted having two arms, but catheter 400 may include any suitable number of arms.

Sensor circuitry 420 may be mechanically coupled to catheter 400, may be physically contacting a distal end of catheter 400, or may be distal to the distal end of catheter 400. In some examples, the assembly includes introducer 402 and a sensing device including sensor circuitry 420, a conductive element, and a sensing element (both not shown in FIG. 5), where the conductive element communicatively couples sensor circuitry 420 and the sensing element. The sensing device may include a distal portion which includes sensor circuitry 420 and a portion of the conductive element. The distal portion of the sensing device is intended to be external to a patient when in use. The sensing device may also include a proximal portion (not shown in FIG. 5) which may include the sensing element and a portion of the conductive element. The proximal portion of the sensing device is intended to be inside the patient when in use. Sensor circuitry 420 may be an example of sensor circuitry 20 of FIG. 1. Sensor circuitry 420 may include electronic, optoelectronic, and/or optical components and be configured to process a signal from the sensing element via the conductive element. The sensing element may be an example of sensing element 21 of FIG. 1 and be configured to sense a parameter of interest in a fluid.

Introducer 402 is configured to facilitate introduction of a conductive element in a catheter lumen (e.g., lumen 32 or drainage lumen 34 shown in FIG. 2), and, therefore, is configured (e.g., sized and shaped) to be received in the catheter lumen. In addition, introducer 402 defines an introducer lumen (not shown in FIG. 5) configured to receive the conductive element and the sensing element. For example, introducer 402 can be a sheath or the like. Introducer 402 may be structurally configured to be relatively flexible, pushable, and relatively kink- and buckle-resistant, so that it may resist buckling when a pushing force is applied to a relatively distal portion of introducer 402 to advance introducer 402 into a catheter lumen of catheter 400. Kinking and/or buckling of introducer 402 may hinder a clinician's efforts to push the elongated body proximally.

In some examples, introducer 402 is stiffer than catheter 400 so as to facilitate the insertion of introducer 402 into or removal of introducer 402 from a catheter lumen. In addition or instead, in some examples, introducer 402 is stiffer than the conductive element. In some examples, introducer 402 is constructed of a polymer, such as, but not limited to, polyamide. In some examples, introducer 402 includes one or more coatings, such as a hydrophilic coating or a lubricious coating on an exterior surface of introducer 402 configured to facilitate the insertion of introducer 402 into or removal of introducer 402 from a catheter lumen. The lubricious coating may be configured to reduce static friction and/kinetic friction between introducer 402 and the catheter lumen as introducer 402 is advanced through or retracted from the catheter lumen.

In some examples, introducer 402 also includes a handle, tab, or the like to enable a user to better grip introducer 402, e.g., to insert introducer 402 into a catheter lumen (e.g., lumen 32 or drainage lumen 34 of FIG. 2) of catheter 400 and/or to removing introducer 402 from the catheter lumen. In the example shown in FIG. 5, the handle is shown as a ring 404.

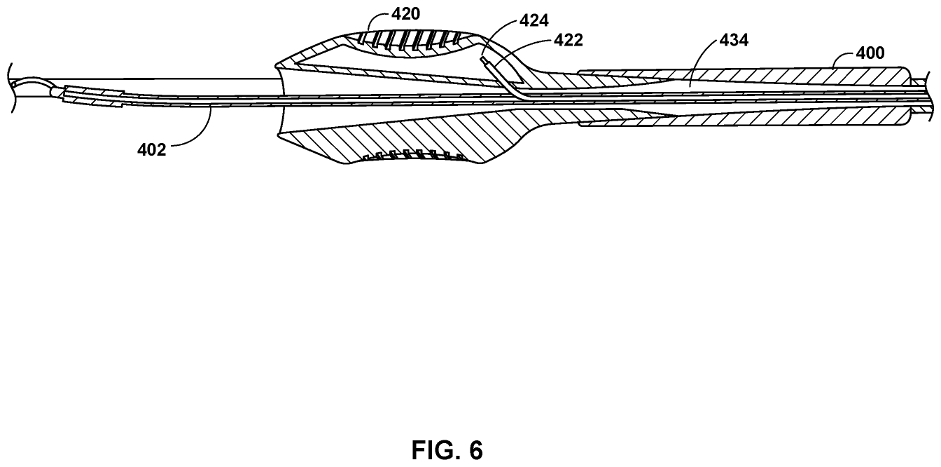

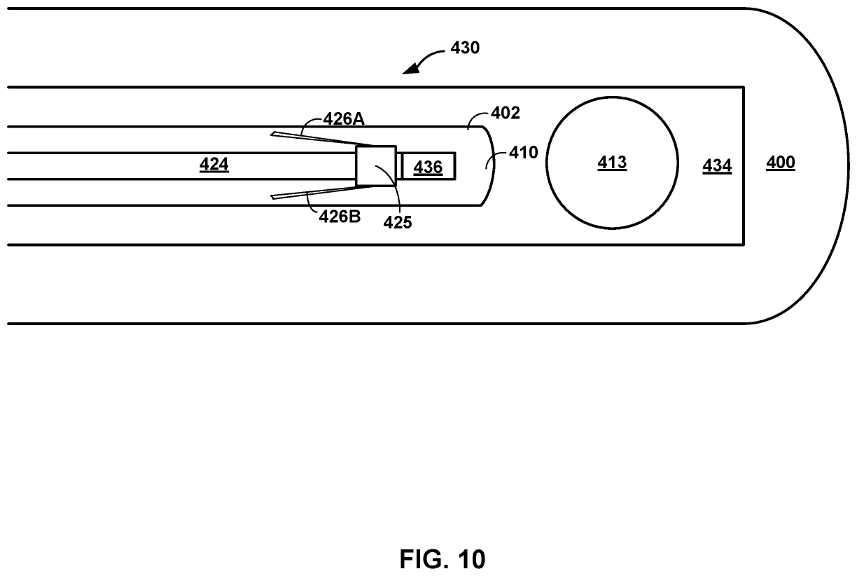

FIG. 6 is a conceptual cross-sectional diagram of an example distal portion of a catheter having an introducer inserted within a catheter lumen defined by the catheter. The cross-section is taken through a longitudinal axis of catheter 400, which is also aligned with the longitudinal axis of introducer 402 and the sensing device in FIG. 6. The sensing device includes sensor circuitry 420, conductive element 424, and a sensing element 436 (FIG. 10). Introducer 402 is depicted partially inserted within catheter lumen 434 of catheter 400.

Introducer 402 is configured to facilitate the introduction of a sensing element (not shown in FIG. 6) and conductive element 424 into the catheter lumen. In some examples, the sensing element is communicatively coupled to conductive element 424, such that a signal or other parameter generated by the sensing element may be transmitted to sensor circuitry 420 via conductive element 424. In some examples, the sensing element is proximal to conductive element 424. In some examples, a distal end of conductive element 424 is communicatively coupled to sensor circuitry 420.

Once the sensing element and/or conductive element 424 are at an appropriate location within catheter lumen 434 (e.g., proximal to anchoring member 18 (of FIG. 1)) and/or after catheter 400 is positioned within the patient as desired, a clinician may distally retract introducer 402 from catheter lumen 434, e.g., to remove introducer 402 from catheter lumen 434 and from around the sensing device (e.g., from around conductive element 424 and/or the sensing element). In this manner, introducer 402, may be used to help the introduction of the sensing element and conductive element 424 within catheter lumen 434 prior to a medical procedure, such that kidney function may be monitored during the medical procedure, and in some examples, thereafter.

In some examples, conductive element 424 and the sensing element is at least partially inside an introducer lumen (not shown in FIG. 6) of introducer 402 before a clinician uses the assembly including the introducer 402 and a sensor device including sensor circuitry 420, conductive element 424 and the sensing element. In other words, the assembly may be pre-assembled with conductive element 424 and the sensing element inside the introducer lumen of introducer 402. In other examples, a clinician may introduce the sensing device into introducer 402 on site, e.g., prior to the medical procedure.

In some examples, the assembly includes rigid member 422. Rigid member 422 extends radially outwards from conductive element 424. Rigid member 422 may be configured to at an angle from introducer 402 that is within an allowable bend angle of conductive element 424. For example, the angle may be in the range of 10-40 degrees. Rigid member 422 may be of lengths 3 mm or greater. In some examples, the length of rigid member 422 may be limited so as to not restrict the bending of the catheter or so as to not protrude outside of the external surface of sensor circuitry 420. Rigid member 422 is configured to help physically separate introducer 402 from conductive element 424 when a clinician withdraws introducer 402 from catheter lumen 434. As discussed in further detail below, rigid member 422 is configured to open a longitudinal surface of introducer 402 as introducer 402 is retracted relative to the sensing device, e.g., as introducer 402 is retracted from the sensing device and out of catheter lumen 434.

Rigid member 422 is relatively rigid to enable rigid member 422 to remain structurally sound as introducer is retracted relative to rigid member 422. In some examples, rigid member 422 includes a needle, such as a hollow needle. Rigid member 422 may be formed from any suitable material, such as, but not limited to, a polymer or a metal.

Rigid member 422 has any suitable dimension that enables it to extend away from conductive element 424 and through a wall thickness of introducer 402, such that rigid member 422 can engage with part of the wall of introducer 402 to help split a longitudinal surface of introducer 402. Splitting of introducer 402 in this manner may better enable introducer 402 to be retracted and physically separated from conductive element 424 without substantially moving the proximal portion of sensing device from a target position in the patient.

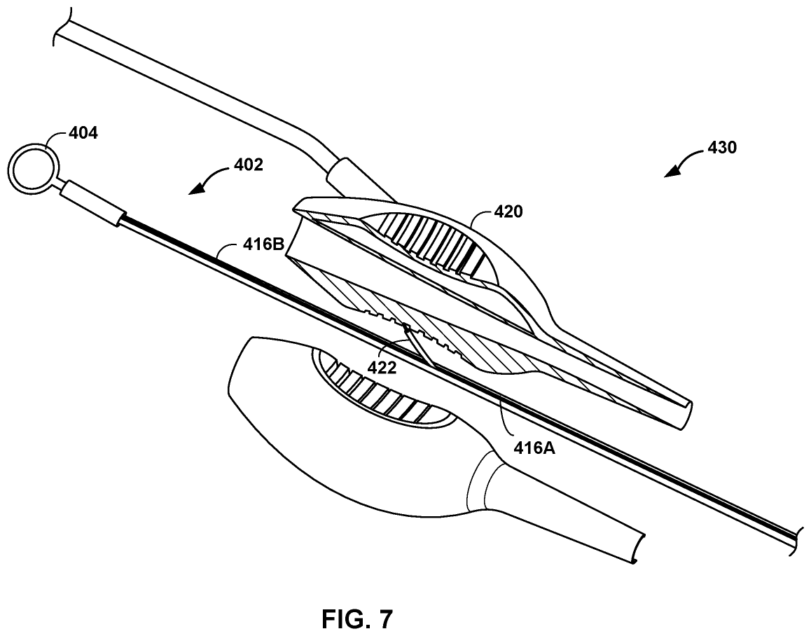

FIG. 7 is a conceptual diagram illustrating an example exploded view of a distal portion of the assembly of FIG. 5. Assembly 430 includes introducer 402, a sensor device including sensor circuitry 420, conductive element 424 (of FIG. 6), a sensing element (not shown in FIG. 7), and rigid member 422. In some examples, assembly 430 may be pre-assembled, e.g., at a manufacturing facility, such that introducer 402 is already inserted through sensor circuitry 420 prior to a clinician inserting introducer 402 into the lumen of catheter 400 (FIGS. 5-6). In other examples, the clinician may insert the sensor device in introducer 402 during the medical procedure or as part of the preparation for the medical procedure.

In some examples, assembly 430 has a suitable length for accessing a portion of catheter lumen 434 (FIG. 6) proximal to anchoring member 18 (of FIG. 1). Conductive element 424 (FIG. 6) and the sensing element may have any appropriate size that may enable conductive element 424 and the sensing element to be located within the introducer lumen. In some examples, conductive element 424 and the sensing element may have a diameter in the range of 250-500 micrometers. In some examples, introducer lumen 410 may have a diameter on the order of 1 mm and introducer 402 may have an outer diameter on the order of 2.4 mm. Assembly 430 may have any appropriate size that may enable at least a portion of assembly 430 to be inserted or to be housed within catheter lumen 434.

In some examples, introducer 402 is pre-split and configured to be opened by rigid member 422, which in some examples is located at least partially located inside sensor circuitry 420 (of FIGS. 5-6) and/or at least partially in the introducer lumen defined by introducer 402. For example, the split may extend in a longitudinal direction of introducer 402 from rigid member 422 or from a positioning distal to rigid member 422 to or towards a proximal end of introducer 402. In some examples, a portion of conductive element 424 is located in a rigid member lumen defined by rigid member 422.

Once assembly 430 has been at least partially inserted into a catheter lumen of catheter 400 and sensing element 436 (FIG. 10) and/or conductive element 424 are at an appropriate location within the catheter lumen, a clinician may retract introducer 402 using ring 404 (or other gripping structure) and remove introducer 402 from catheter lumen 434 (which can, be, for example, lumen 32 or drainage lumen 34 (both of FIG. 1)). Split 416 of introducer 402 may be opened by rigid member 422 as introducer 402 is retracted relative to conductive element 424 and in a distal direction (away from sensing element 436). Thus, rigid member 422 is configured to separate introducer 402 from conductive element 424 leaving conductive element 424 and sensing element 436 in place within catheter lumen 434. In other words, when introducer 402 is removed from catheter lumen 434, conductive element 424 and sensing element 436 are not removed from catheter lumen 434 and sensing element 436 and a portion of conductive element 424 remain in catheter lumen 434.

Rigid member 422 may be mechanically coupled to the sensing device and be configured to open a longitudinal surface of introducer 402 as introducer 402 is retracted relative to the sensing device. Rigid member 422 may be configured to protect conductive element 424 from buckling or being removed from catheter lumen 434 as introducer 402 is removed from the catheter lumen. Thus, movement of introducer 402 in the distal direction may cause rigid member 422 to open split 416 in a longitudinal direction.

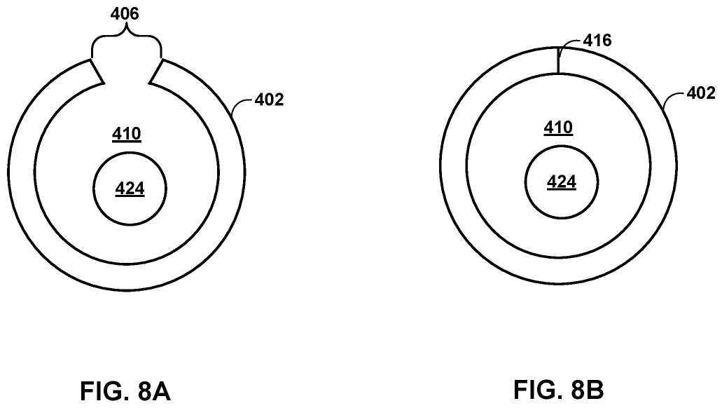

FIGS. 8A and 8B are alternative planar views (e.g., end views) of an example introducer 402. As shown in FIGS. 8A and 8B, introducer 402 defines an introducer lumen 410 which is configured to substantially enclose conductive element 424 (and the sensing element) when being inserted into catheter lumen 434 (FIG. 6) of catheter 400 (FIGS. 5-6). In some examples, introducer 402 may be pre-split. In other words, introducer 402 may include a split or cutaway or be configured to be split. This is represented as cutaway 406 in FIG. 8A. While cutaway 406 is shown as the absence of material of introducer 402, in some examples, rather than cutaway 406, introducer 402 may include split 416 as shown in FIG. 8B. Split 416 may extend from an outer surface of introducer 402 to an inner surface (defining introducer lumen 410) of introducer 402. In some examples, split 416 may extend partially from either the outer surface of introducer 402 towards the inner surface of introducer 402 or from the inner surface of introducer 402 towards the outer surface of introducer 402.

In some examples, cutaway 406 (or split 416) extend along an entire length of introducer 402, i.e., from a proximal end to a distal end of introducer 402. This may enable introducer 402 to be removed from around conductive element 424 in a lateral direction (through a side of introducer). For example, as shown in FIG. 6, a distal portion of conductive element 424 may extend into a part of a sensing device that includes sensor circuitry 420. As a result, introducer 402 and conductive element 424 are not coaxial along their entire lengths, such that introducer 402 cannot be removed from around conductive element 424 by withdrawing introducer 402 past a distal end of conductive element 424.

Rigid member 422 is configured such that rigid member 422 extends through cutaway 406 (or split 416) and helps maintains conductive element 424 within catheter lumen 434 (not shown in FIGS. 8A and 8B) when introducer 402 is retracted or removed from catheter lumen 434 of catheter 400 (of FIG. 6). Rigid member 422 may be configured to, when a clinician retracts introducer 402 from the catheter lumen, open split 416. Even if cutaway 406 or split 416 are predefined openings through a wall of introducer 402, rigid member 422 is configured to further open cutaway 406 or split 416 to help open up introducer lumen 410 and help disengage introducer 402 from conductive element 424.

In FIG. 7, an open portion of split 416 is shown as distal portion of split 416B and an unopened portion of split 416 is shown as proximal portion of split 416A. Thus, as the clinician pulls on ring 404 to retract or remove introducer 402 from the catheter lumen, rigid member 422 opens split 416 such that distal portion of split 416B that is distal to rigid member 422 has been opened by rigid member 422 and proximal portion of split 416A that is proximal to rigid member 422 has not yet been opened by rigid member 422. Alternatively, in an example where introducer 402 includes cutaway 406, rigid member 422 may contact sides of cutaway 406.

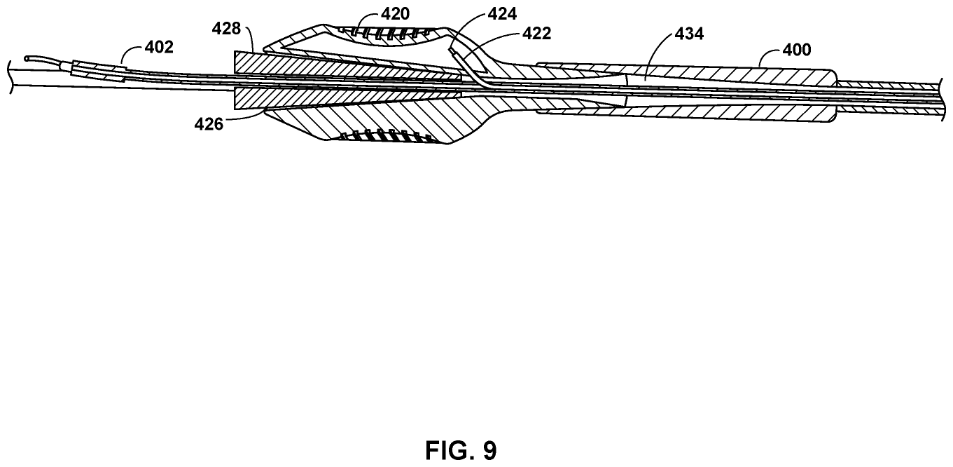

FIG. 9 is a conceptual diagram of a distal portion of an example assembly inserted within a catheter lumen of a catheter. A portion of introducer 402 may be located within sensor circuitry lumen 426 defined by sensor circuitry 420. A proximal portion of assembly 430 may be inserted into catheter lumen 434 of catheter 400. As can be seen, rigid member 422 is located at least partially within sensor circuitry 420 and penetrates into a longitudinal split defined by introducer 402 (e.g., split 416 or cutaway 406 of FIGS. 8A-8B, not shown in FIG. 9).

To ensure that introducer 402 stays in physical contact with rigid member 422 and rigid member 422 does not dislodge from split 416 or cutaway 406 when introducer 402 is retracted or removed from catheter lumen 434, in some examples conical insert 428 is included in assembly 430 (FIG. 7). Conical insert 428 may have a relatively conical shape. In some examples, conical insert 428 may have another shape. Conical insert 428 may be configured to hold introducer 402 in place such that rigid member 422 remains in cutaway 406 or split 416 (or other longitudinal split) while introducer 402 is retracted or removed, until a proximal end of introducer 402 is distal to rigid member 422. In some examples, conical insert 428 is configured to be inserted into sensor circuitry 420. For example, conical insert 428 may be configured to be inserted into the sensing device and be configured to help maintain contact between rigid member 422 and introducer 402 during at least a portion of the retraction of introducer 402 from catheter lumen 434. For example, conical insert 428 may be configured to provide support to introducer 402 to maintain an end of rigid member 422 within cutaway 406 or split 416 of introducer 402. Once introducer 402 is removed from catheter lumen 434, a clinician may also remove conical insert 428 from sensor circuitry 420. In some examples, assembly 430 does not include conical insert 428.

FIG. 10 is a conceptual diagram of a proximal portion of an example assembly within a catheter lumen of a catheter. Catheter 400 is shown defining catheter lumen 434. In the example of FIG. 10, catheter lumen 434 is fluidically coupled to fluid opening 413 which may provide access to catheter lumen 434 to a fluid, such as urine in a bladder of a patient. A proximal portion of introducer 402 may be inserted, by a clinician, into catheter lumen 434. While the example of FIG. 10 shows assembly 430 in catheter lumen 434, assembly 430 may be located in any suitable lumen of catheter 400.

Conductive element 424 may be communicatively coupled to sensing element 436. For example, conductive element 424 may be an optical fiber configured to conduct optical signals from sensing element 436 to sensor circuitry 420 (of FIGS. 5-7, and 9). In some examples, conductive element 424 may be a plastic optical multimode fiber. Plastic optical multimode fiber may be more flexible and less brittle than a glass fiber and may have a higher numerical aperture thereby facilitating conductive element 424 to acquire more light from sensing element 436, in examples where sensing element 436 is an optical sensing element. In another example, conductive element 424 may be an electrical wire configured to conduct electrical signals from sensing element 436 to sensor circuitry 420. In some examples, sensing element 436 may be configured to sense an oxygen content of a fluid.

Conductive element 424 is configured to communicatively couple sensor circuitry 420 to sensing element 436, e.g., is configured to conduct an optical signal or an electrical signal between sensing element 436 and sensor circuitry 420. In some examples, the conductive element may be an optical fiber or an electrical wire.

Conductive element 424 and sensing element 436 may be within introducer lumen 410 of introducer 402. It may be desirable to prevent sensing element 436 from contacting a surface defining catheter lumen 434 when sensing a parameter of interest in a fluid in catheter lumen 434, as contacting the surface defining catheter lumen 434 may cause noise in the sensed signal. In some examples, cuff 425 having tines 426A-426B may be attached to conductive element 424, for example, using an adhesive. Tines 426A-426B located on a proximal portion a sensing device and be configured to prevent sensing element 436 from contacting the surface defining catheter lumen 434 when tines 426A-426B are deployed. In some examples, tines 426A-426B may be configured to reduce direct contact between conductive element 424 and a sidewall of catheter 400 defining catheter lumen 434 when conductive element 424 and tines 426A-426B are positioned in catheter lumen 434. In some examples, tines 426A-426B may be made from thin sheet metal, such as stainless steel, titanium, memory metal, or other non-corrosive metals and may be made using etching, laser cutting or punching techniques followed by a forming to a circular shape fitting to conductive element 424, such as cuff 425 with tines 426A-426B protruding outward from cuff 425. In some examples, when tines 426A-426B are deployed, tines 426A-426B form a 30 degree angle from conductive element 424. In some examples, the length of tines 426A-426B is on the order of about 1.75 mm.

In some examples, conductive element 424 (or sensing element 436) may include a plurality of tines, such as tines 426A-426B. Tines 426A-426B may be configured to expand radially outward relative to conductive element 424 from an undeployed configuration to a deployed configuration. Tines 426A-426B may be configured to be in the undeployed configuration when tines 426A-426B are within introducer lumen 410 (as shown in FIG. 10) and to be in the deployed configuration when tines 426A-426B are outside of introducer lumen 410 and within a catheter lumen (e.g., catheter lumen 434). For example, tines 426A and 426B may be undeployed and contained within introducer lumen 410 when conductive element 424 is inside of introducer lumen 410. In some examples, tines 426A and 426B may be collapsed against conductive element 424 (or sensing element 436) when inside of introducer lumen 410. While two tines are shown in FIG. 10, the number of tines may be greater than two.

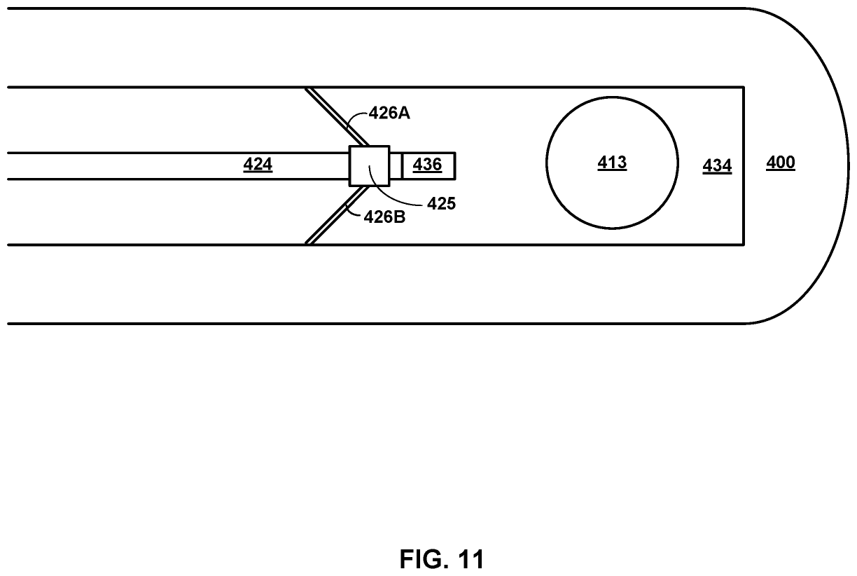

FIG. 11 is a conceptual diagram of an example conductive element and sensing element within a catheter lumen after an introducer catheter is retracted from the catheter lumen. When introducer 402 is retracted from catheter lumen 434, tines 426A-426B may deploy like an umbrella, as shown, to contact the surface defining catheter lumen 434 and hold sensing element 436 in the center (or near the center) of catheter lumen 434 so as to prevent or impede sensing element 436 from contacting a surface defining catheter lumen 434.

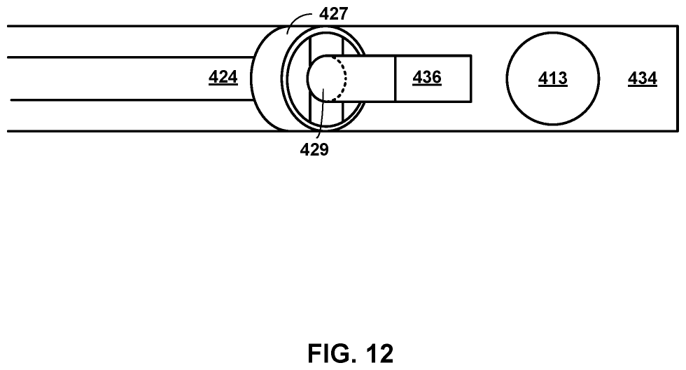

FIG. 12 is a conceptual diagram illustrating an example ring configured to help prevent a sensing element from contacting a catheter surface defining a catheter lumen. In some examples, rather than using tines 426A-426B to prevent or impede sensing element 436 from contacting a surface defining catheter lumen 434, a system can include ring 427 to reduce or even prevent sensing element 436 from contacting the surface defining catheter lumen 434. For example, ring 427 may be located on a proximal portion a sensing device and be configured to reduce direct contact between the conductive element and a sidewall of the catheter defining the catheter lumen when the conductive element and the plurality of tines are positioned in the catheter lumen. Ring 427 may include opening 429 sized to permit conductive element 424 to fit there within. The outer diameter of ring 427 may be sized to fit within catheter lumen 434. In some examples, ring 427 may be attached to conductive element 424 using an adhesive.

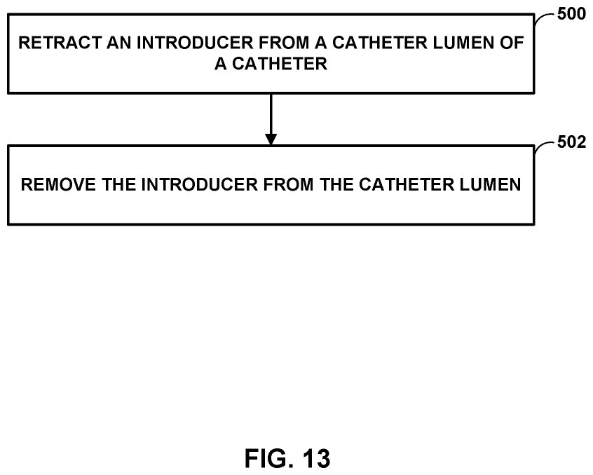

FIG. 13 is a flow diagram illustrating assembly usage techniques of this disclosure. A clinician may retract an introducer from a catheter lumen of a catheter (500).

For example, the clinician may pull on ring 404 to retract introducer 402 from catheter lumen 434 of catheter 400. In some examples, introducer 402 defines introducer lumen 410 configured to receive at least a portion of conductive element 424. In some examples, introducer 402 is configured to be inserted into the catheter lumen of the catheter (e.g., catheter lumen 434 of catheter 400) while the at least the portion of conductive element 424 is in introducer lumen 410.

The clinician may remove the introducer from the catheter lumen (502). For example, the clinician may continue to pull on ring 404 to completely remove the introducer from catheter lumen 434 of catheter 400. At least a portion of conductive element 424 and sensing element 436 may remain inside the catheter lumen (e.g., catheter lumen 434 of catheter 400) even after the introducer is removed from the catheter lumen.

In some examples, rigid member 422 is mechanically coupled to the sensing device and is configured to open a longitudinal surface of introducer 402 as introducer 402 is retracted relative to the sensing device. In some examples, rigid member 422 is a hollow needle and conductive element 424 may be partially within a lumen defined by rigid member 422.

In some examples, the clinician inserts introducer 402 into the catheter lumen (e.g., catheter lumen 434). In some examples, introducer 402 defines split 416 from an outer surface of introducer 402 to introducer lumen 410, the split extending in a longitudinal direction of introducer 402. In some examples, rigid member 422 is configured to be received in split 416 as introducer 402 is retracted relative to the sensing device.

In some examples, rigid member 422 is further configured to physically contact sides of split 416 (or cutaway 406) and wherein retracting introducer 402 at least partially opens split 416 in introducer 402. In some examples, retracting the introducer causes a plurality of tines (e.g., tines 426A-426B) on a proximal portion of the sensing device to expand radially outward relative to conductive element 424 from an undeployed configuration to a deployed configuration. In some examples, the plurality of tines are configured to be in the undeployed configuration when the plurality of tines are within introducer lumen 410 and to be in the deployed configuration when the plurality of tines are outside of introducer lumen 410 and within the catheter lumen (e.g., catheter lumen 434).

In some examples, a clinician removes conical insert 428 from the sensing device. In some examples, conical insert 428 is configured to prevent the sides of split 416 from losing contact with rigid member 422 during at least a portion of the retracting of introducer 402 from the catheter lumen (e.g., catheter lumen 434).

Any of the techniques or examples described herein may be used alone or in combination with one or more other techniques or examples. These techniques may improve the ability more accurately sense oxygen content in a fluid than locating an oxygen sensor on a distal portion of a catheter or distal to a distal end of the catheter, as the oxygen content in the fluid may change as the fluid transits through the catheter. The techniques of this disclosure may provide an alternative location for a fiberoptic system than the drainage lumen of the Foley catheter. These techniques may improve the ability more accurately sense oxygen content in a fluid as drainage lumens may become clogged, for example, with blood, tissue, or other substances that may be in the bladder of the patient. Furthermore, by providing a location within the interior of the Foley catheter, there need not be an oxygen sensor element on body of catheter, thereby making the catheter easier to insert as there is only silicon in contact with the urethra of a patient.

The techniques described in this disclosure, including those attributed to sensor circuitry 20, sensing element 21, processing circuitry 200, communication circuitry 206, and UI 204 or various constituent components, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated or discrete logic circuitry. The term “processor” or “processing circuitry” may generally refer to any of the foregoing logic circuitry, alone or in combination with other logic circuitry, or any other equivalent circuitry.

Such hardware, software, firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

When implemented in software, the functionality ascribed to the systems, devices and techniques described in this disclosure may be embodied as instructions on a computer-readable medium such as RAM, ROM, NVRAM, EEPROM, FLASH memory, magnetic data storage media, optical data storage media, or the like. The instructions may be executed to support one or more aspects of the functionality described in this disclosure.

This disclosure includes the following non-limiting examples.

Example 1. An assembly comprising: a sensing device configured to sense a parameter of interest in a fluid, the sensing device comprising sensor circuitry at a distal portion, a sensing element at a proximal portion, and a conductive element communicatively coupling the sensor circuitry and the sensing element; an introducer defining an introducer lumen configured to receive at least a portion of the conductive element of the sensing device, the introducer being configured to be at least partially inserted into a catheter lumen of a catheter while the at least the portion of the conductive element is in the introducer lumen; and a rigid member mechanically coupled to the sensing device and configured to open a longitudinal surface of the introducer as the introducer is retracted relative to the sensing device.

Example 2. The assembly of example 1, wherein the introducer defines a split from an outer surface of the introducer to the introducer lumen, the split extending in a longitudinal direction of the introducer, and wherein the rigid member is configured to be received in the split.

Example 3. The assembly of example 2, wherein the rigid member is further configured to physically contact sides of the split and at least partially open the split when the introducer is retracted from the catheter lumen and retracted relative to the sensing element.

Example 4. The assembly of example 3, wherein the rigid member comprises a hollow needle.

Example 5. The assembly of any of examples 1-4, wherein the proximal portion of the sensing device comprises a plurality of tines configured to expand radially outward relative to the conductive element from an undeployed configuration to a deployed configuration, the plurality of tines being configured to be in the undeployed configuration when the plurality of tines are within the introducer lumen and to be in the deployed configuration when the plurality of tines are outside of the introducer lumen and within the catheter lumen.

Example 6. The assembly of example 5, wherein the plurality of tines are configured to reduce direct contact between the conductive element and a sidewall of the catheter defining the catheter lumen when the conductive element and the plurality of tines are positioned in the catheter lumen.

Example 7. The assembly of any of examples 1-4, wherein the proximal portion of the sensing device comprises a ring configured to reduce direct contact between the conductive element and a sidewall of the catheter defining the catheter lumen when the conductive element and the plurality of tines are positioned in the catheter lumen.

Example 8. The assembly of any of examples 1-7, wherein the introducer is stiffer than the conductive element.

Example 9. The assembly of any of examples 1-8, wherein the conductive element comprises an electrical wire or an optical fiber.

Example 10. The assembly of example 9, wherein the conductive element comprises an optical fiber and wherein the parameter of interest is oxygen.

Example 11. The assembly of any of examples 1-10, further comprising a conical insert configured to be inserted into the sensing device and being configured to help maintain contact between the rigid member and the introducer during at least a portion of the retraction of the introducer from the catheter lumen.

Example 12. The assembly of any of examples 1-11, wherein the introducer comprises a hydrophilic coating on an exterior surface of the introducer.

Example 13. The assembly of any of examples 1-12, wherein the introducer comprises polyamide.

Example 14. The assembly of any of examples 1-13, further comprising the catheter.

Example 15. A method comprising: retracting an introducer from a catheter lumen of a catheter, the introducer defining an introducer lumen configured to receive at least a portion of a conductive element of a sensing device, the introducer being configured to be at least partially inserted into the catheter lumen while the at least the portion of the conductive element is in the introducer lumen, wherein a rigid member is mechanically coupled to the sensing device and is configured to open a longitudinal surface of the introducer as the introducer is retracted relative to the sensing device; and removing the introducer from the catheter lumen.

Example 16. The method of example 15, further comprising inserting the introducer into the catheter lumen while the at least the portion of the conductive element is in the introducer lumen.

Example 17. The method of example 15 or example 16, wherein the introducer defines a split from an outer surface of the introducer to the introducer lumen, the split extending in a longitudinal direction of the introducer, and wherein the rigid member is received in the split as the introducer is retracted from the catheter lumen and is retracted relative to the sensing device.

Example 18. The method of example 17, wherein the rigid member is further configured to physically contact sides of the split and wherein retracting the introducer at least partially opens the split in the introducer.

Example 19. The method of any of examples 15-18, wherein retracting the introducer causes a plurality of tines on a proximal portion of the sensing device to expand radially outward relative to the conductive element from an undeployed configuration to a deployed configuration, the plurality of tines being configured to be in the undeployed configuration when the plurality of tines are within the introducer lumen and to be in the deployed configuration when the plurality of tines are outside of the introducer lumen and within the catheter lumen.

Example 20. The method of any of examples 15-19, further comprising: removing a conical insert from the sensing device, the conical insert being configured to help maintain contact between the rigid member and the introducer during at least a portion of the retracting of the introducer from the catheter lumen.

Example 21. An assembly comprising: a catheter defining a catheter lumen; a sensing device configured to sense oxygen content in a fluid, the sensing device comprising sensor circuitry at a distal portion, a sensing element at a proximal portion, and an optical fiber communicatively coupled to the sensor circuitry and the sensing element; an introducer defining an introducer lumen configured to receive at least a portion of the optical fiber, the introducer being at least partially disposed within the catheter lumen and being configured to be retracted from the catheter lumen while the at least the portion of the optical fiber is in the introducer lumen; and a rigid member mechanically coupled to the sensing device and configured to open a longitudinal surface of the introducer as the introducer is retracted relative to the sensing device.

Various examples have been described. These and other examples are within the scope of the following claims.

What is claimed is:

1. An assembly comprising:
a sensing device configured to sense a parameter of interest in a fluid, the sensing device comprising sensor circuitry at a distal portion, a sensing element at a proximal portion, and a conductive element communicatively coupling the sensor circuitry and the sensing element;
an introducer defining an introducer lumen configured to receive at least a portion of the conductive element of the sensing device, the introducer being configured to be at least partially inserted into a catheter lumen of a catheter while the at least the portion of the conductive element is in the introducer lumen; and
a rigid member mechanically coupled to the sensing device and configured to open a longitudinal surface of the introducer as the introducer is retracted relative to the sensing device.

2. The assembly of claim 1, wherein the introducer defines a split from an outer surface of the introducer to the introducer lumen, the split extending in a longitudinal direction of the introducer, and wherein the rigid member is configured to be received in the split.

3. The assembly of claim 2, wherein the rigid member is further configured to physically contact sides of the split and at least partially open the split when the introducer is retracted from the catheter lumen and retracted relative to the sensing element.

4. The assembly of claim 3, wherein the rigid member comprises a hollow needle.

5. The assembly of claim 1, wherein the proximal portion of the sensing device comprises a plurality of tines configured to expand radially outward relative to the conductive element from an undeployed configuration to a deployed configuration, the plurality of tines being configured to be in the undeployed configuration when the plurality of tines are within the introducer lumen and to be in the deployed configuration when the plurality of tines are outside of the introducer lumen and within the catheter lumen.

6. The assembly of claim 5, wherein the plurality of tines are configured to reduce direct contact between the conductive element and a sidewall of the catheter defining the catheter lumen when the conductive element and the plurality of tines are positioned in the catheter lumen.

7. The assembly of claim 1, wherein the proximal portion of the sensing device comprises a ring configured to reduce direct contact between the conductive element and a sidewall of the catheter defining the catheter lumen when the conductive element and the plurality of tines are positioned in the catheter lumen.

8. The assembly of claim 1, wherein the introducer is stiffer than the conductive element.

9. The assembly of claim 1, wherein the conductive element comprises an electrical wire or an optical fiber.

10. The assembly of claim 9, wherein the conductive element comprises an optical fiber and wherein the parameter of interest is oxygen.

11. The assembly of claim 1, further comprising a conical insert configured to be inserted into the sensing device and being configured to help maintain contact between the rigid member and the introducer during at least a portion of the retraction of the introducer from the catheter lumen.

12. The assembly of claim 1, wherein the introducer comprises a hydrophilic coating on an exterior surface of the introducer.

13. The assembly of claim 1, wherein the introducer comprises polyamide.

14. The assembly of claim 1, further comprising the catheter.

15. A method comprising:
retracting an introducer from a catheter lumen of a catheter, the introducer defining an introducer lumen configured to receive at least a portion of a conductive element of a sensing device, the introducer being configured to be at least partially inserted into the catheter lumen while the at least the portion of the conductive element is in the introducer lumen, wherein a rigid member is mechanically coupled to the sensing device and is configured to open a longitudinal surface of the introducer as the introducer is retracted relative to the sensing device; and
removing the introducer from the catheter lumen.

16. The method of claim 15, further comprising inserting the introducer into the catheter lumen while the at least the portion of the conductive element is in the introducer lumen.

17. The method of claim 16, wherein the introducer defines a split from an outer surface of the introducer to the introducer lumen, the split extending in a longitudinal direction of the introducer, and wherein the rigid member is received in the split as the introducer is retracted from the catheter lumen and is retracted relative to the sensing device.

18. The method of claim 17, wherein the rigid member is further configured to physically contact sides of the split and wherein retracting the introducer at least partially opens the split in the introducer.

19. The method of claim 15, further comprising:
removing a conical insert from the sensing device, the conical insert being configured to help maintain contact between the rigid member and the introducer during at least a portion of the retracting of the introducer from the catheter lumen.

20. An assembly comprising:
a catheter defining a catheter lumen;
a sensing device configured to sense oxygen content in a fluid, the sensing device comprising sensor circuitry at a distal portion, a sensing element at a proximal portion, and an optical fiber communicatively coupled to the sensor circuitry and the sensing element;
an introducer defining an introducer lumen configured to receive at least a portion of the optical fiber, the introducer being at least partially disposed within the catheter lumen and being configured to be retracted from the catheter lumen while the at least the portion of the optical fiber is in the introducer lumen; and
a rigid member mechanically coupled to the sensing device and configured to open a longitudinal surface of the introducer as the introducer is retracted relative to the sensing device.
